(19) United States
(12) Patent Application Publication
(10) Pub. No.: US 2020/0283730 A1
(43) Pub. Date: Sep. 10, 2020

(54) Systems and Methods for Automated Processing of Signals from Cardiomyocytes in an Unbiased Manner

(71) Applicant: The Board of Trustees of the Leland Stanford Junior University, Stanford, CA (US)

(72) Inventors: Jayakumar Rajadas, Stanford, CA (US); Joseph Wu, Stanford, CA (US); Andrey Victorovich Malkovskiy, Stanford, CA (US)

(73) Assignee: The Board of Trustees of the Leland Stanford Junior University, Stanford, CA (US)

(21) Appl. No.: 16/811,672

(22) Filed: Mar. 6, 2020

Related U.S. Application Data
(60) Provisional application No. 62/814,810, filed on Mar. 6, 2019.

(51) Int. Cl. C12N 5/077; G01N 33/487; C12M 1/36

(52) U.S. Cl. CPC C12N 5/0657; G01N 33/48728; G06T 7/0012; G01N 2500/10; C12M 41/48

(57) ABSTRACT

The present disclosure describes systems and methods capable of automated processing of signals from cardiomyocytes in an unbiased manner. Embodiments of the present disclosure are directed to analyzing signal traces derived from cardiomyocytes that provide more accurate and reliable information. Additional embodiments are directed to assessing disease mechanisms and/or treatment plans for an individual based on cardiomyocyte function, and further embodiments are directed to treating an individual based on cardiomyocyte function.

### CROSS-REFERENCE TO RELATED APPLICATIONS

This application claims priority to U.S. Provisional Application Ser. No. 62/814,810, entitled “Systems and Methods for Automated Processing of Fluorescence Signals from Calcium Transients in an Unbiased Manner” to Rajadas et al., filed Mar. 6, 2019, which is herein incorporated by reference in its entirety.

### FIELD OF THE INVENTION

The present invention relates generally to cellular contractility in cardiomyocytes, including methods to assess contractility data from cardiomyocytes and uses thereof. More particularly, computational methods that analyze peak data arising from cellular contractility that provide more accurate assessment of the peaks created by cardiomyocyte “beats.”

### BACKGROUND OF THE INVENTION

Recent development of techniques to produce iPSC patient-derived cardiomyocytes has allowed screening drugs for certain disease conditions, thus avoiding a clinical study involving actual patients, with all its complications involved. Not only can the iPSC-derived cardiomyocytes be stored and then grown and matured just when they are required for an experiment, but also one can conduct drug safety studies in parallel with drug efficacy testing, significantly speeding the path of novel drug development.

The benefit of measurements of single cells is much higher consistency in experimental conditions, to which the cell is subjected. In addition, certain calculations are more straightforward and reliable, for example, the amount of drug intake per cell, change of dimensions, among other measurements.

Traditionally, Ca²⁺ imaging has been utilized to observe single cell behavior. Sometimes, usually for the sake of yet higher consistency, patch clamp experiments are performed, although they have vastly lower throughput and are more difficult to perform.

### SUMMARY OF THE INVENTION

Systems and methods for automated processing of signals from cardiomyocytes in an unbiased manner in accordance with embodiments of the invention are disclosed.

In one embodiment, a method for processing signals from calcium transients includes obtaining cardiomyocyte data comprising one dimensional trace data, identifying a peak in the one dimensional trace data, and fitting the peak to a shape function.

In a further embodiment, the method includes identifying peak features based on the shape functions.

In another embodiment, the cardiomyocyte data is obtained from at least one of the following: electrocardiography, fluorescence, and atomic force microscopy.

In a still further embodiment, the peaks are identified by a local maxima above a threshold.

In still another embodiment, the threshold is set to 0.7*(Yₘₐₓ−Yₘᵢₙ)+Yₘᵢₙ.

In a yet further embodiment, the fit shape function is a Voigt function.

In yet another embodiment, the shape function is fit using a Levenberg-Marquadt algorithm.

In a further embodiment again, the method includes identifying a key peak feature from the shape function.

In another embodiment again, the key peak feature is selected from the group consisting of peak start, peak 50%, 90% decay, peak 50% width, peak 90% width, maximum rising rate, maximum falling rate, pulse T₅₀, standard deviation of beat interval, standard deviation of beat rate, T₅₀, amplitude (ΔF/F₀), average maximum rising rate, and average maximum decaying rate.

In a further additional embodiment, a method to treat an individual based on cardiomyocyte function includes obtaining a cardiomyocyte from an individual, treating the cardiomyocyte with a drug, obtaining a measurement of cardiomyocyte function of the cardiomyocyte, assessing treatment efficacy of the drug based on the measurement of cardiomyocyte function, and treating the individual with the drug.

In another additional embodiment, the cardiomyocyte is an iPSC derived cardiomyocyte.

In a still yet further embodiment, the measurement of cardiomyocyte function is obtained from at least one of the following: electrocardiography, fluorescence, and atomic force microscopy.

In still yet another embodiment, the assessing step is accomplished by identifying a peak in the one dimensional trace data and fitting the peak to a shape function.

In a still further embodiment again, the peaks are identified by a local maxima above a threshold.

In still another embodiment again, the threshold is set to 0.7*(Yₘₐₓ−Yₘᵢₙ)+Yₘᵢₙ.

In a still further additional embodiment, the fit shape function is a Voigt function.

In still another additional embodiment, the shape function is fit using a Levenberg-Marquadt algorithm.

In a yet further embodiment again, the assessing step further includes identifying a key peak feature from the shape function.

In yet another embodiment again, the key peak feature is selected from the group consisting of peak start, peak 50%, 90% decay, peak 50% width, peak 90% width, maximum rising rate, maximum falling rate, pulse T₅₀, standard deviation of beat interval, standard deviation of beat rate, T₅₀, amplitude (ΔF/F₀), average maximum rising rate, and average maximum decaying rate.

In a yet further additional embodiment, the obtaining a measurement of cardiomyocyte function is a baseline measurement of cardiomyocyte function and further comprising obtaining a measurement of cardiomyocyte function of the treated cardiomyocytes.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

These and other features and advantages of the present invention will be better understood by reference to the following detailed description when considered in conjunction with the accompanying drawings where:

FIG. 1 illustrates a method for analyzing cardiomyocyte traces in accordance with various embodiments.

FIGS. 2A-2C illustrate peak identification, function fitting, and feature identification of cardiomyocyte beats in accordance with various embodiments.

FIGS. 3A-3F illustrate output of exemplary software identifying peaks and peak features in accordance with various embodiments.

FIG. 4 illustrates a method to identify disease mechanisms and/or effective drugs in accordance with various embodiments.

FIGS. 5A-5L illustrate results from calcium trace experiments in accordance with various embodiments.

FIGS. 6A-6K illustrate results from atomic force microscopy measurements in accordance with various embodiments.

### DETAILED DESCRIPTION

The embodiments of the invention described herein are not intended to be exhaustive or to limit the invention to precise forms disclosed. Rather, the embodiments selected for description have been chosen to enable one skilled in the art to practice the invention.

Turning now to the drawings, Human induced pluripotent stem cells (iPSCs) enable robust generation of cardiomyocytes (iPSC-CMs) in large quantities. iPSC-CMs from patients carrying inherited disease-causing mutations in combination with site-specific gene editing via CRISPR/Cas9 present an alternative model to study molecular disease mechanisms and drug effects. Many embodiments enable deeper and more accurate profiling of disease-specific differences in cardiomyocyte contraction profiles. Many embodiments will be able to more accurately profile any one-dimensional measurements, including electrocardiography (EKG/ECG), fluorescence, and atomic force microscopy (AFM). Certain embodiments will use patient-derived iPSC-CMs. A number of embodiments enable this profiling by providing by providing adaptive computing of signal peaks for different Ca²⁺ flux or force levels in iPSC-CMs, as well as analysis of Ca²⁺ sparks. Application of certain embodiments allows for both Ca²⁺ trace and AFM recording parameters, such as half-maximal rising rate, decay time, and duration of contraction with an automated background noise detection. Improved analysis of Ca²⁺ traces and AFM recordings can identify disease mechanisms to guide better treatment protocols and/or better decisions for drugs, such that iPSC-CMs from an individual can be used to identify which drugs will work to treat the person's disease and/or understand the underlying mechanism of their genetic mutation.

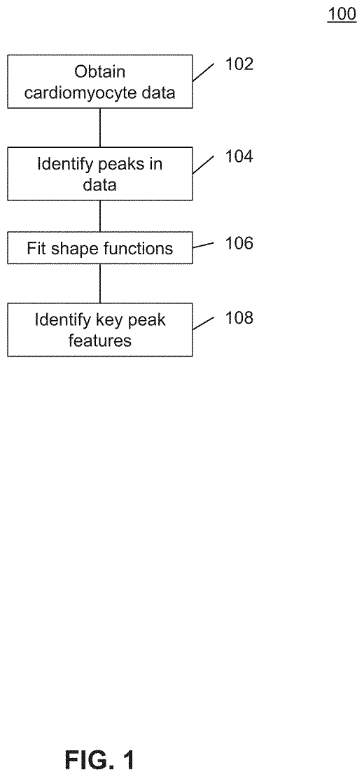

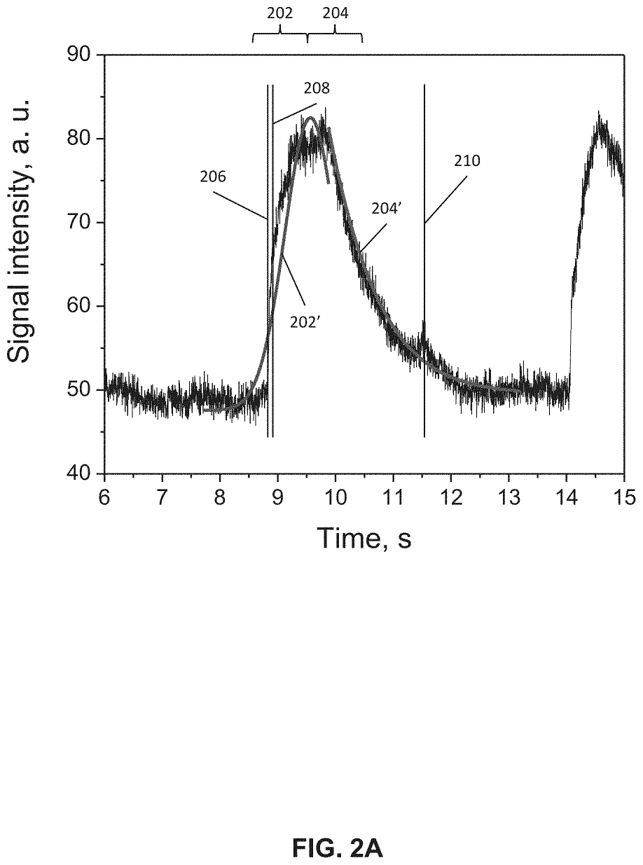

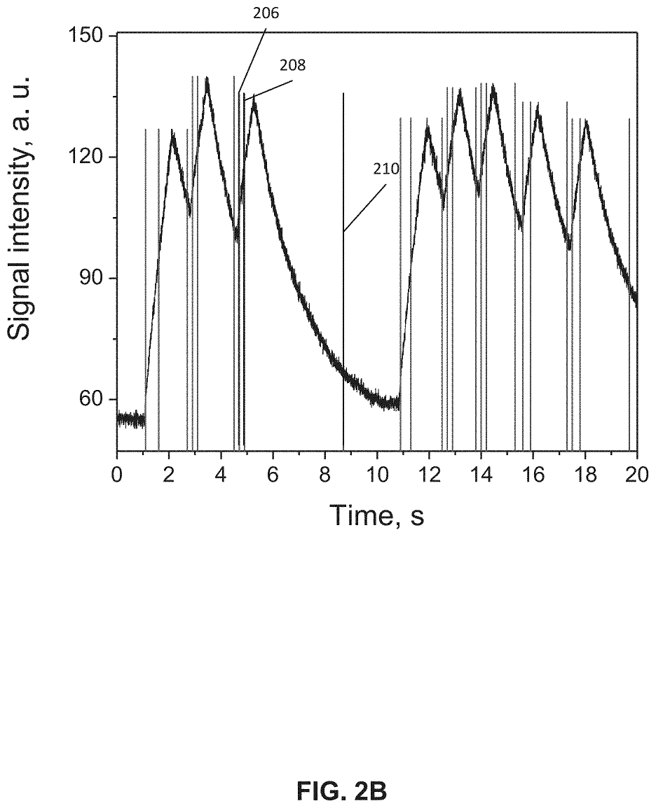

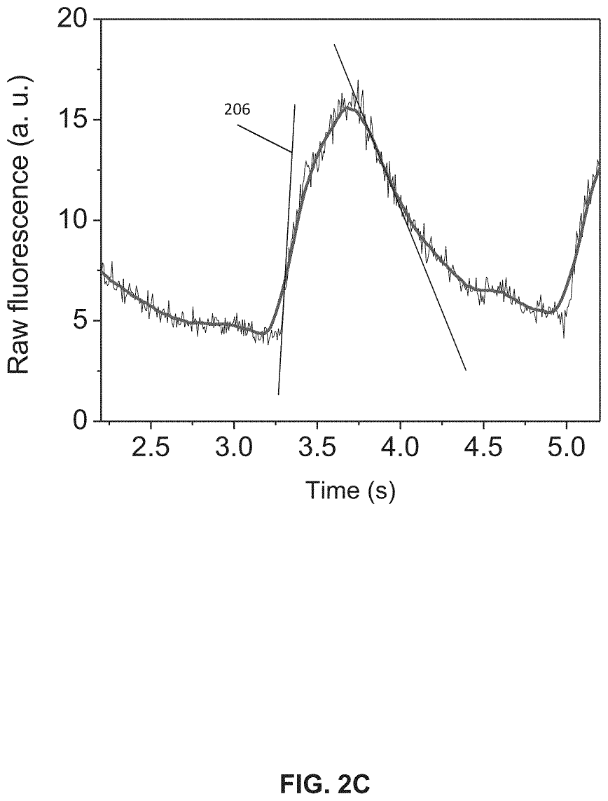

Turning to FIG. 1, a method 100 for processing signals from calcium transients is illustrated. At step 102, cardiomyocyte data is obtained. In certain embodiments, the data is obtained from a saved data source, such as local media, hard drive, or server, while some embodiments will obtain the cardiomyocyte data as a live stream of data being acquired. The data is obtained from any applicable source, and many embodiments will be able to more accurately profile any one-dimensional measurements, including electrocardiography (EKG/ECG), fluorescence, and atomic force microscopy (AFM). Typically, a cardiomyocyte “beat” spans approximately 8 seconds, and a typical beat profile in Ca²⁺ imaging is illustrated FIG. 2A. As seen in FIG. 2A, many beats have a rising side 202 and decay side 204.

Returning to FIG. 1, at step 104 of many embodiments, peaks within the cardiomyocyte are identified. In certain embodiments, the peaks are identified by assessing local minima an maxima. Certain embodiments will detect approximate peak positions by finding local maxima above a threshold. Some embodiments will set the threshold to 0.7*(Yₘₐₓ−Yₘᵢₙ)+Yₘᵢₙ, indicating a threshold of 70% of the distance between the minimum and maximum Y values. Further embodiments will allow the threshold to be set to any value, such as 50%, 60%, 75%, 80%, 85%, 90%, and 95% of the distance between the minimum and maximum Y values. Various embodiments will use maxima and minima information to calculate rate of beating and intervals between beats.

Turning back to FIG. 1, numerous embodiments will fit peaks to shape functions at step 106. Peaks in many embodiments are non-symmetric, including in Ca²⁺ imaging and AFM height-time traces. As illustrated in FIG. 2A, peak fit models are illustrated in an exemplary embodiment, including a rising side fit 202′ and a decay side fit 204′. Certain embodiments will begin peak fitting with the decay side, and additional embodiments will assume that a majority of decay sides 204 resemble a Voigt function, which is a combination of a Lorentzian and a Gaussian profile. Voigt functions have a peak center, which allows the fit to “anchor” to a data trace. Further, starting peak fitting step 106 with the decay portion of the beat provides more data points due to the extended shape of the profile and, consequently, better fit accuracy. Additional embodiments will fit to a Voigt function using a Levenberg-Marquadt algorithm. Fitting the peak to a function can also include weights, such that certain weights will minimize noise or effects due to overlapping peaks in the data.

In many embodiments, a full trace correction with a polynomial background accounts well for overall fluorescence signal decay over time, while in other embodiments, an adaptive local correction based on a running average accounted effectively for short-scale background fluctuations. In certain embodiments, the kernel size is set to average distance between peaks for optimum performance.

A variety of embodiments will use other profiles for peak fitting. However, Lorentzian profiles, when used alone for approximation, often overestimate the decay curve profile (e.g., the curve falls off slower than actual), while Gaussian profiles, when used alone for approximation, often underestimate the decay curve profile. Furthermore, neither Lorentzian nor Gaussian profiles work well to describe a rising side 202 shape. Additionally, while exponential decay functions are able to approximate decay side 204 sides, exponential functions say nothing of the start of a peak, thus do not allow for proper estimates of 50% and 90% decay times.

Returning to FIG. 1, numerous embodiments will identify key peak features at Step 108. Returning to FIG. 2A, Voigt fit profiles are plotted rising 202′ and decay 204′ sides are labeled following a peak start 206 for an exemplary embodiment. Additionally, in this embodiment, peak 50% 208 and 90% decay 210 are further illustrated as key features of a peak. Many embodiments will determine peak start 206 by a variety of methods, including by a fast rise of the curve, or, in mathematical terms, by a peak in the derivative, which has been smoothened by adjacent averaging to account for noise (e.g., FIG. 2A). For data that is less noisy, it is possible to detect the peak start 206 by local minimum of a smoothened dip before the peak (e.g., FIG. 2B). Many embodiments will allow for any number of features understood to those of skill in the art, such that the 10% peak, 25% peak, 75% peak, 50% decay, 75% decay, and any other percentage can be identified. A number of embodiments will be able to calculate width of a peak at 10% height, 25% height, 75% height or any other percentage of interest.

In many embodiments, the decay side 204′ has more datapoints and allows a more accurate determination of peak widths. Various embodiments determine peak 50% width as follows: the time position of the middle value (Yₘᵢₙ+Yₘₐₓ)/2 of the decay curve Voigt fit is determined, then the first matching time position is found on the rising side, if approached from the peak center, the width is then simply the distance between the two time positions. Various embodiments will determine peak 90% width via similar methods. An attempt to calculate the mid-point of the rising side without prior knowledge of decay side generally gives inconsistent results.

Turning to FIG. 2C, the maximum rising rate 212 and maximum falling rate 214 are determined in certain embodiments from the steepest slopes of the rising and decay sides. As can been seen from FIG. 2C, estimating the slope from a smoothened profile results in severely underestimated values, particularly for the rising side. Instead, many embodiments will use adaptive basis-spline fitting with the number of knots or control points adjusted based on the local noise in the data. Using a basis-spline fitting method, the slope is minimally affected. In addition to maximum rising and falling rates, rising and falling rates can be identified for any other part of the peak.

Further embodiments will determine additional parameters, including at least one of the following: pulse T₅₀, standard deviation of beat interval, standard deviation of beat rate, T₅₀, amplitude (ΔF/F₀), average maximum rising rate, and average maximum decaying rate.

It should be noted that the steps of method 100 can be performed in different a different order, simultaneously and/or omitted, as necessary for a particular task. One of skill in the art would understand that a person would be able to augment method 100 to allow for optimization and/or performance of method 100 for a particular use. Furthermore, certain embodiments of method 100 will be embodied as non-transitory machine readable media containing processor instructions, where execution of the instructions causes a processor to execute the steps of method 100. Further, embodiments will be directed to systems including a processor and memory that are specially purposed to perform the steps of method 100.

### Software Embodiments

A number of embodiments are directed to software capable of executing the method 100 illustrated in FIG. 1. These embodiments also allow for additional parameters or controls to be implemented. For example, in some embodiments, the software will include file path entry or the ability to point to the data source and/or live stream.

Additional embodiments will further allow a user to specify particular background correction on the raw data obtained. Background correction in some embodiments can be selected from Fourier transform band stop, manual thresholding, flatten advanced, flattening by polynomial curve subtraction, or a combination thereof. Fourier transform band stop can be enabled to clean periodic noise in raw data, while manual thresholding can be used to override any auto-determined threshold. Flatten advanced can be used to subtract a running average, because better results from peak and feature detection may be better when the running average is close to average peak spacing.

Certain embodiments will allow for additional parameters to be input, depending on the input raw data, including camera frame rate, column number, jump multiplier, number of data to be analyzed, discard small peaks, delete first X peaks. Camera frame rate can be used to convert camera data into a time domain (e.g., into seconds). For example, if using camera data, the frame rate can be used to convert the data into seconds. Column number allows for data with multiple traces in a single file with multiple columns; this parameter can be used to select which column or columns to analyze. A jump multiplier is generally set to approximate the expected distance between peaks in seconds, where typically, the jump multiplier is set between 70-90% of an expected distance between peaks. The number of data to be analyzed sets for each trace analyzed as a separate set in the output file. Discard small peaks sets a lower limit threshold, where any peaks not reaching that threshold will be ignored in analysis. Finally, delete first X peaks allows for the first X number of peaks to be discarded when the initial peaks in the raw data possesses bad peaks (e.g., start of measurement, unstable system operation, etc.).

Output of mane embodiments will include peak and feature identification, such as discussed in method 100 of FIG. 1. In certain embodiments, this output is generated as an analog table identifying each peak position and the features (e.g., peak height, peak 50%, etc.). However, additional output in certain embodiments will include graphical representation of the raw data (such as if the trace data is input as analog data) and/or peak identification after thresholding (e.g., to remove small peaks).

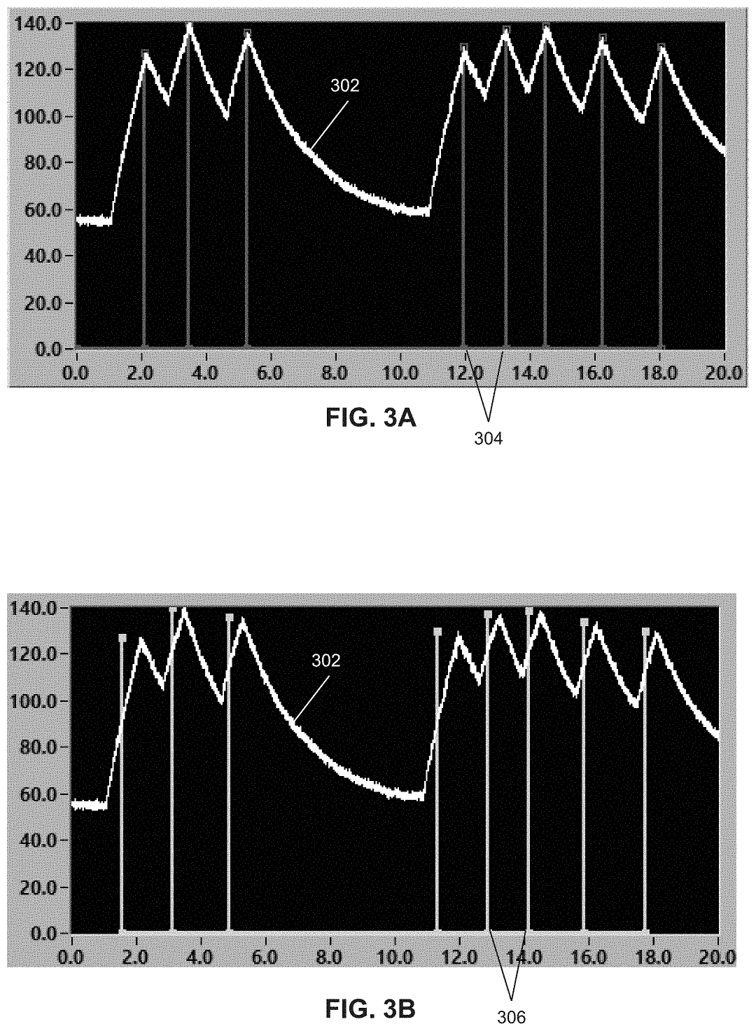

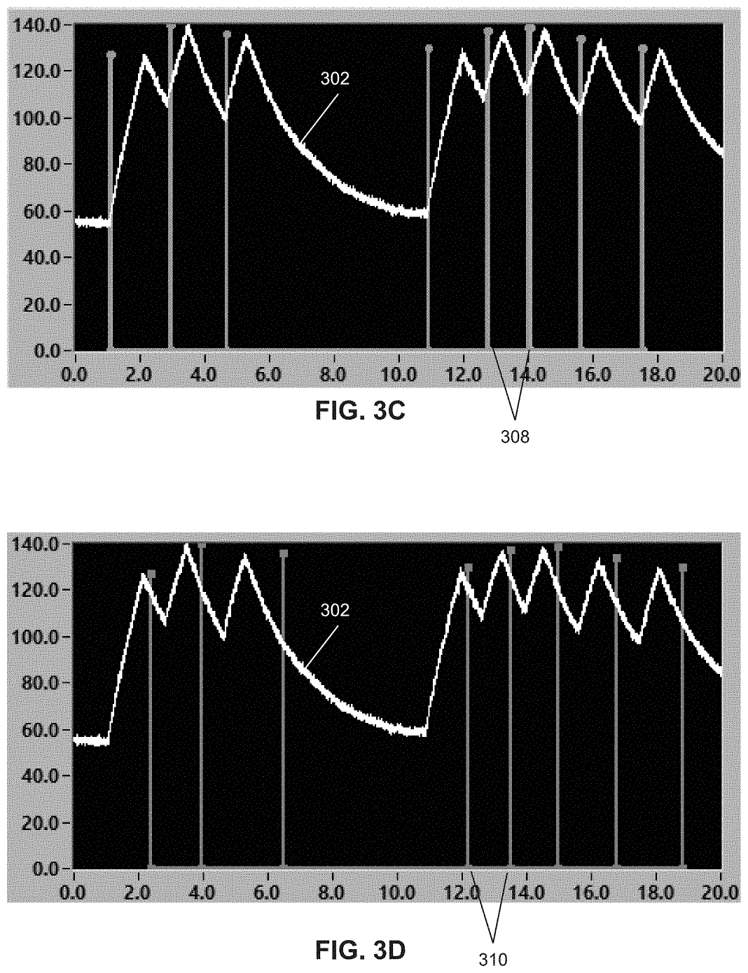

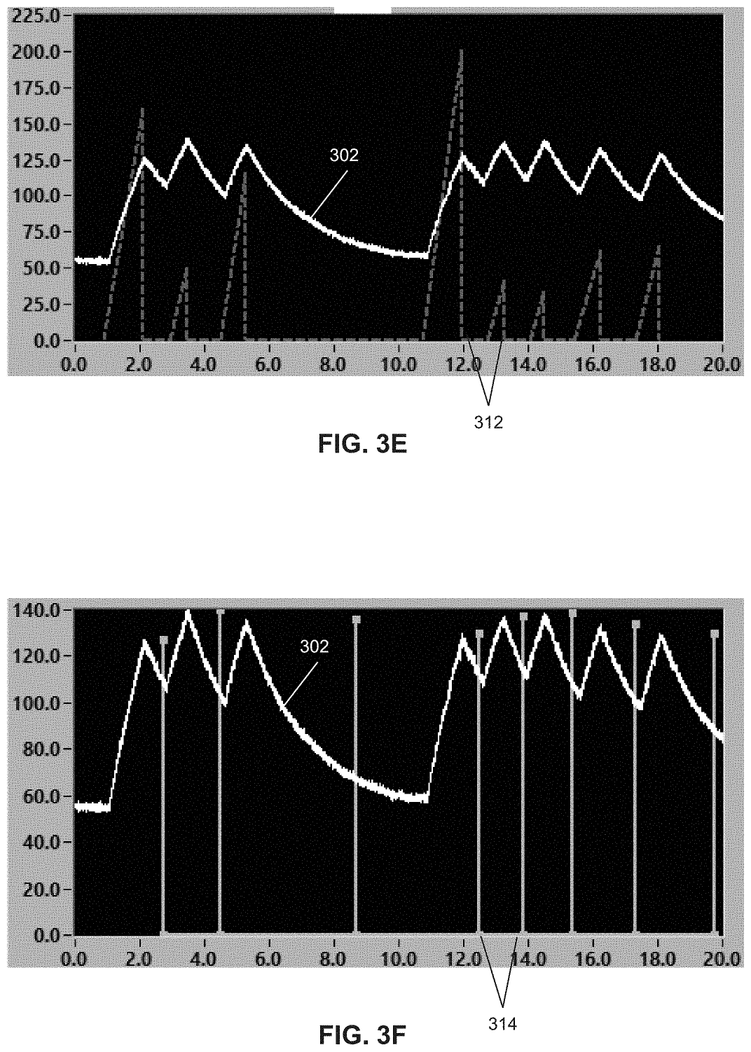

Turning to FIGS. 3A-3F, examples of output from an embodiment are illustrated to show peak and feature identification in accordance with many embodiments. Specifically, FIGS. 3A-3F illustrate the raw data 302 along with peak position 304 (FIG. 3A), peak 50% 306 (FIG. 3B), peak start 308 (FIG. 3C), 50% decay 310 (FIG. 3D), max rise rate 312 (FIG. 3E), and 90% decay 314 (FIG. 3F).

### Methods to Identify Disease Mechanisms and/or Effective Drugs that can be Used in Treating an Individual

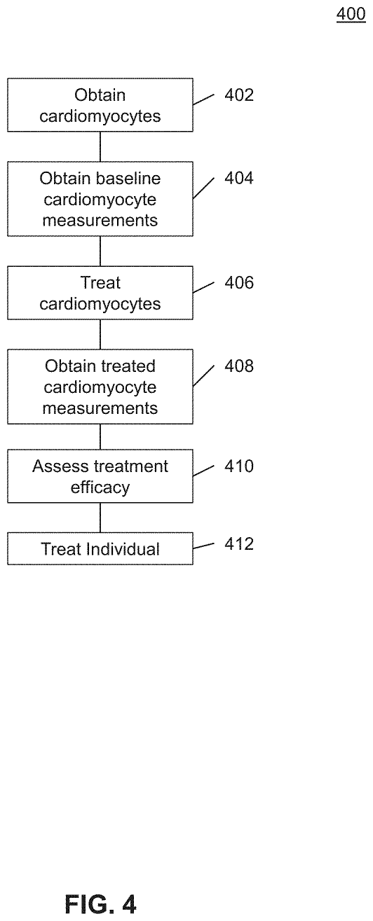

Turning to FIG. 4, a method 400 of numerous embodiments to identify disease mechanism, effective drugs for an individual suffering from cardiac dysfunction, and/or treating an individual is illustrated. At step 402, cardiomyocytes are obtained. Certain embodiments will obtain the cardiomyocytes as iPSC derived cardiomyocytes from an individual. In some embodiments, the cardiomyocytes possess a genetic mutation that correlates with abnormal cardiac function and/or cardiac disease. Some embodiments will create mutations in cardiomyocytes to assess the effect on cardiomyocyte function. In the embodiments that create mutations, the mutations can be novel or previously unknown mutations, while some will recreate known mutations to assess effect of the mutation on cardiomyocyte function. Certain mutation embodiments will use a CRISPR system (e.g., CRISPR/Cas9), while other embodiments will use other mutagenic systems (e.g., chemical or biochemical mutagenesis) to create these mutations.

At step 404, some embodiments will obtain baseline measurements of the obtained cardiomyocytes. In certain embodiments, the measurements are one-dimensional measurements. In certain embodiments, the measurements are obtained from electrocardiography (EKG/ECG), fluorescence (including Ca²⁺ imaging), and/or atomic force microscopy (AFM).

At step 406, the cardiomyocytes are treated in some embodiments. The treatment can be with a drug known to or suspected of affecting cardiomyocyte function in vivo. In certain embodiments, multiple drugs can be tested and/or multiple doses or dosing methods will be tested against the cardiomyocytes. Certain embodiments for identifying disease mechanisms will treat the cardiomyocytes with agonists on the cardiomyocytes to alter function of the cardiomyocytes and possibly identify underlying mechanism of a disease.

At step 408, measurements of treated cardiomyocytes are obtained. This step will be performed in various embodiments using the same measurement system as used in step 404.

In step 410, certain embodiments will assess the effect of the drug or treatment on the cardiomyocytes using methods for peak analysis and feature identification. In certain embodiments, step 410 utilizes methods, including method 100 in FIG. 1, to assess peaks in the measurements. Step 410 can identify which drug, dose, or dosing method that is more effective in treating the cardiomyocytes.

At step 412 of treating methods, the patient is treated in accordance with the drug dose or dosing system identified to be effective in previous steps.

It should be noted that the steps within method 400 can be performed in a different order than illustrated in FIG. 4. Additionally, certain embodiments will omit certain steps that may not be necessary for operation of particular functionality, while additional embodiments will duplicate certain steps, combine certain steps, and/or perform certain steps simultaneously, depending on need for a particular application.

### EXEMPLARY EMBODIMENTS

Although the following embodiments provide details on certain embodiments of the inventions, it should be understood that these are only exemplary in nature, and are not intended to limit the scope of the invention.

### Example 1: Assessing Ca2+ Handling Data in DCM Patient-Derived iPSC-CMs with Voigt-Basis Spline Fitting Detects Disease-Specific Phenotypes

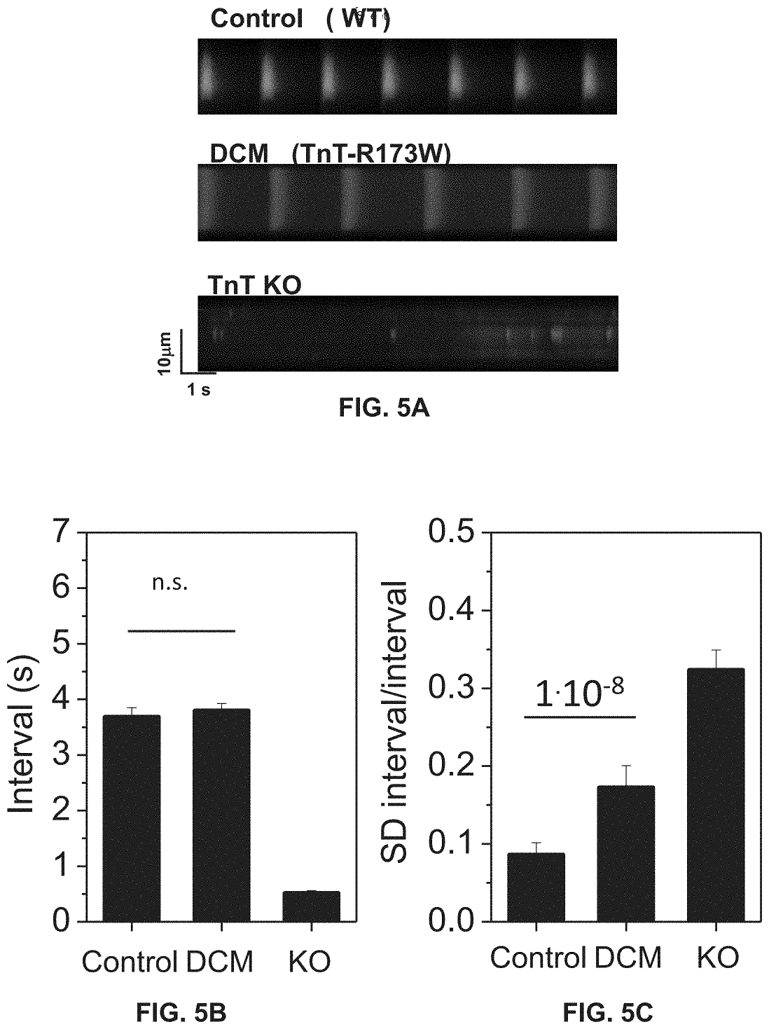

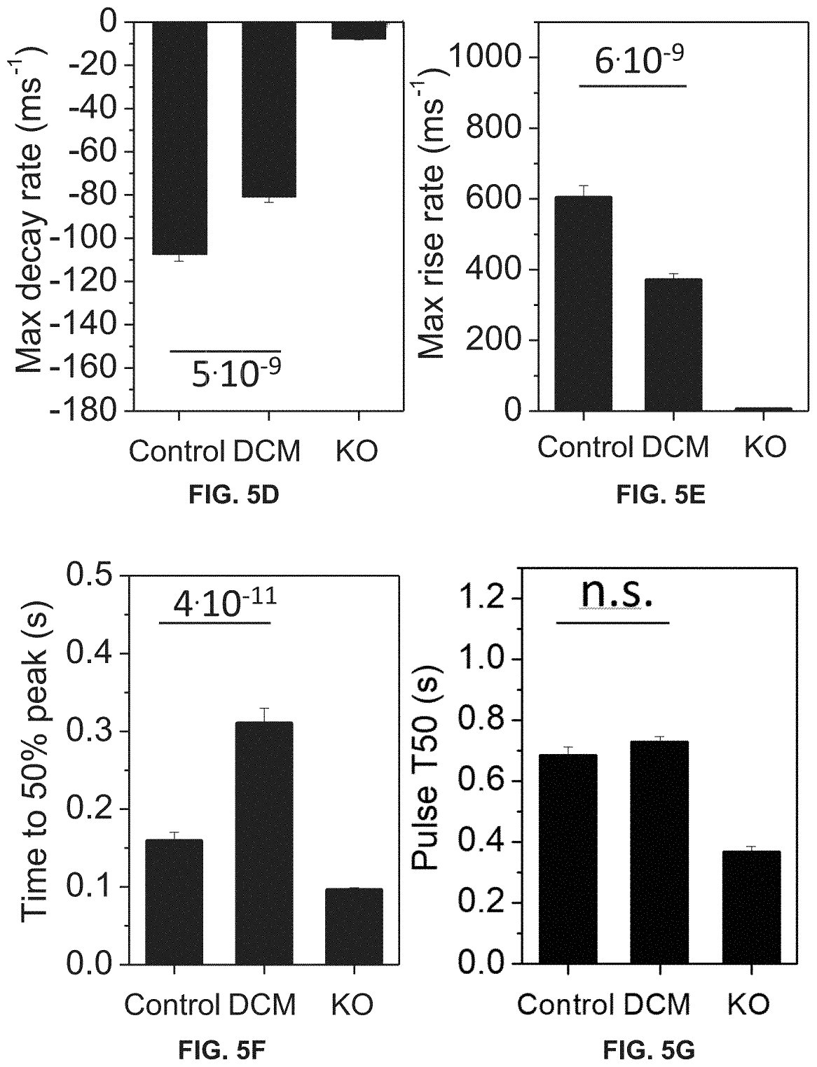

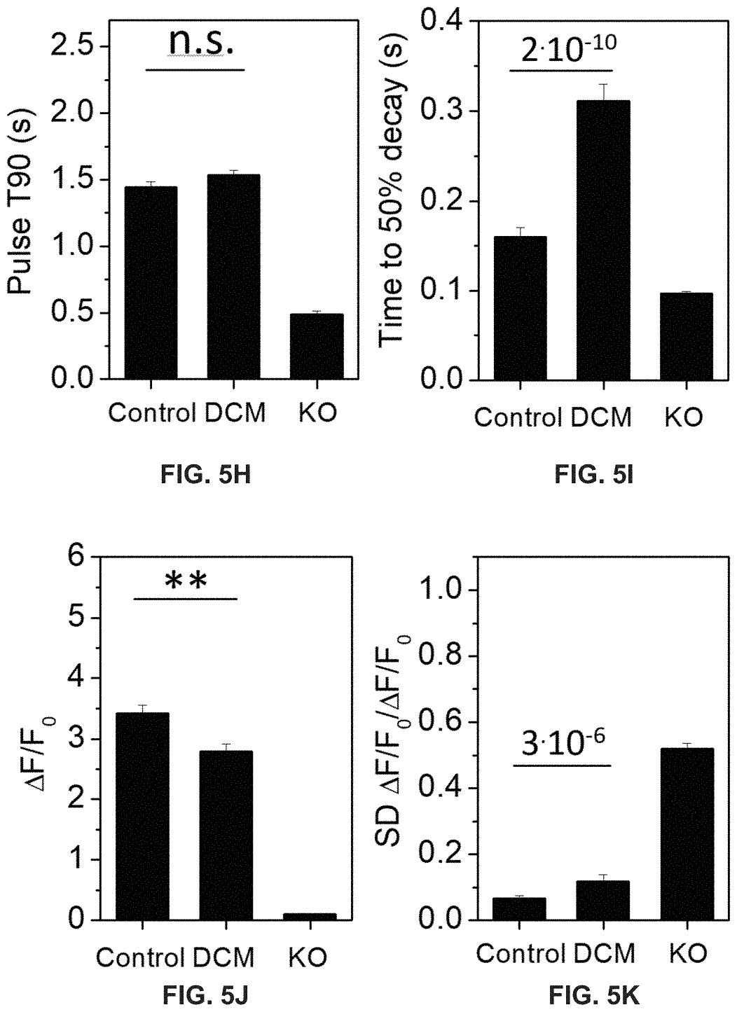

METHODS: DCM patient-specific and healthy WT control iPSC-CMs were employed to measure Ca²⁺ handling via live confocal imaging in Fluo-4 AM-loaded cells (FIG. 5A). Next, Ca²⁺ traces were analyzed by Voigt-basis spline fitting, such as described elsewhere herein. Significantly increased time-to-peak and decreased amplitude (defined here as ΔF/F₀) and decay time _{T} (FIG. 5I) were found. The peak shape change is computed with different Ca²⁺ flux levels. The half-maximal and maximal rising rate, as well as contraction duration were calculated as additional parameters.

Additionally, arrhythmic events in DCM iPSC-CMs and WT controls were analyzed. Arrhythmic events were calculated as a standard deviation of distances between beats for every given beating sequence. Variation of interval length between individual beats was plotted as standard deviation of 5 min rate intervals measured.

RESULTS: FIGS. 5A-5L illustrate analysis of Calcium (Ca²⁺ ) transients in DCM and TnT KO iPSC-CMs, as well as healthy controls. DCM patient-specific iPSC-CMs display disturbed Ca²⁺ handling. Shown are representative traces (FIG. 5A); interval time between beats (FIG. 5B); the standard deviation time from beat to beat (SD Interval) (FIG. 5C); maximum decay rate (FIG. 5D); maximum rise rate (FIG. 5E); time to 50% peak (FIG. 5F); pulse T50 (FIG. 5G); pulse T90 (FIG. 5H); time to 50% decay (FIG. 5I); transient amplitude shown as average ΔF/F0 (FIG. 5J); standard deviation of transient amplitude (FIG. 5K); and time to 50% decay (FIG. 5L). Bar graphs show averages for control (n=2 cell lines) and DCM-TnT-R173W (n=3 cell lines). *P<0.05, **P<0.01 as calculated by Student's t-test. Data are shown as mean±sem.

CONCLUSION: Importantly, the new analysis method allows for the calculation of parameters of the negative control cardiomyocytes, which did not present typical “Ca flux” transients, but instead showed the commonly called “Ca sparks”—low intensity spikes in Ca²⁺ signal, barely above baseline.

### Example 2: Extending Voigt-Basis Spline Fitting Analysis to single-cell AFM Force Measurements Enables More Accurate Profiling of Disease Phenotypes

BACKGROUND: To test if improved analysis of certain embodiments can be applied also to contractile force measurements, non-invasive single-cell AFM in DCM patient-specific iPSC-CMs and WT controls was utilized.

METHODS: This embodiment obtained Ca²⁺ flux and AFM recordings in line with known procedures, including those discussed herein. Trace analysis was performed in accordance with methods described herein and compared between the Ca²⁺ flux and the AFM recordings.

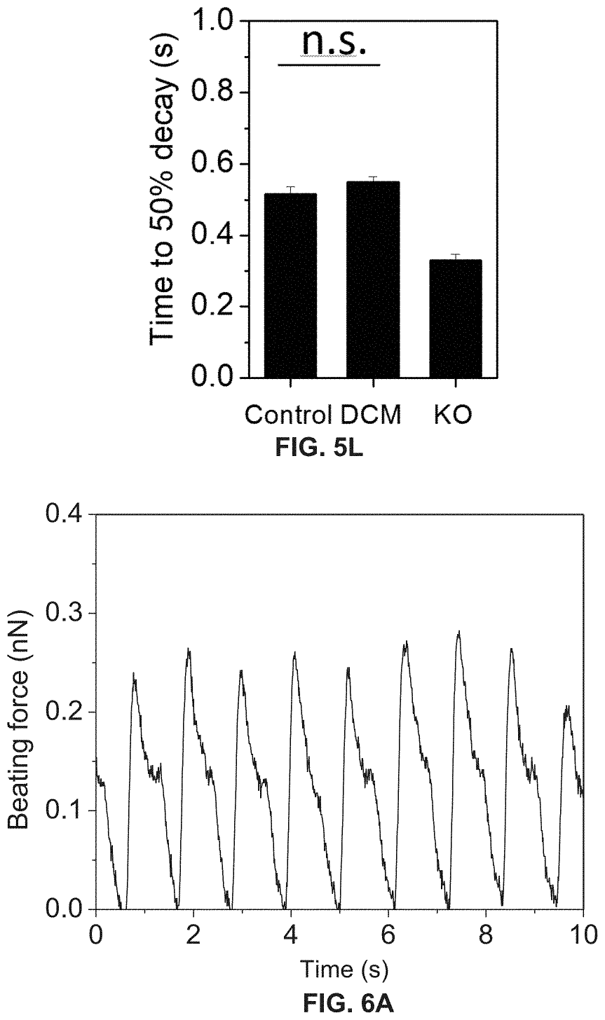

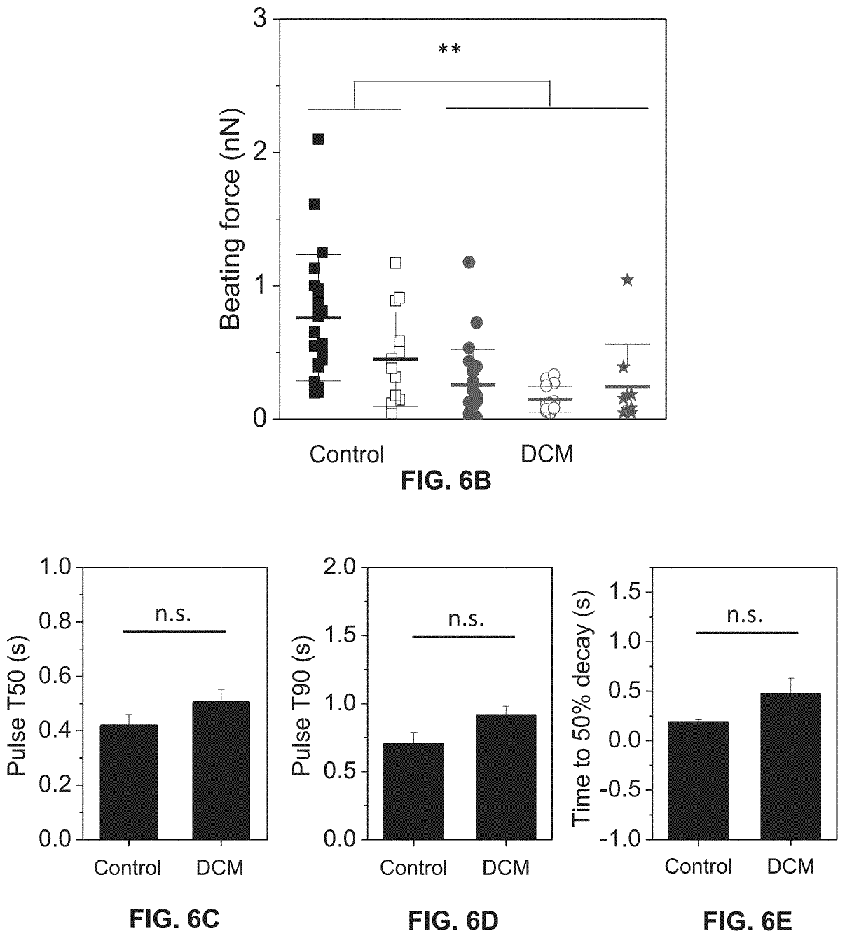

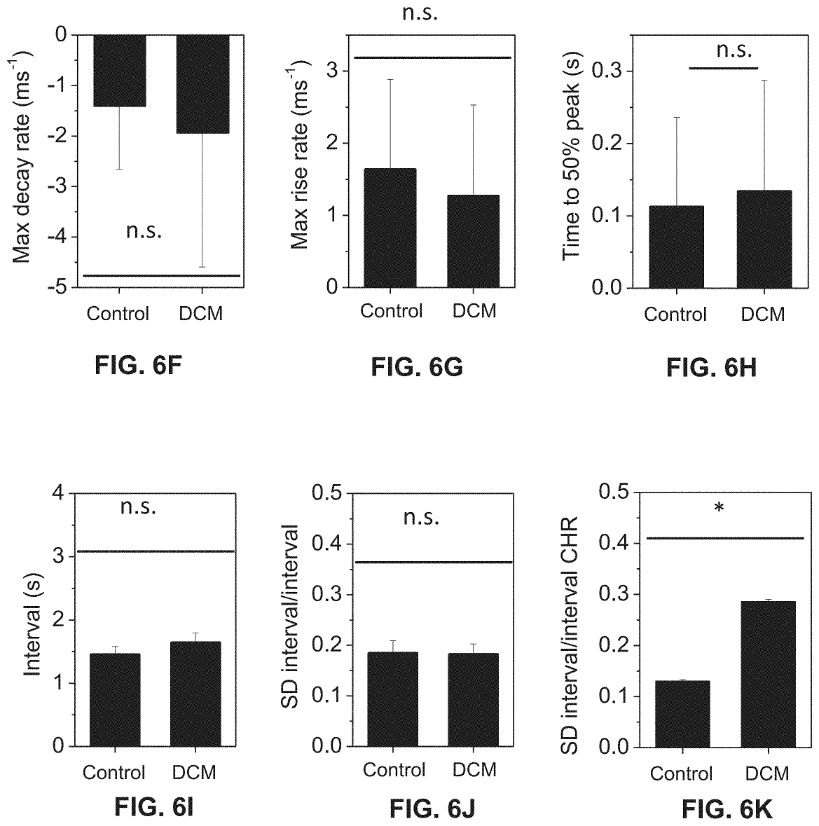

RESULTS: In line with prior reports, significantly decreased beating force in DCM iPSC-CMs were found (FIGS. 6A-6B). Isogenic TnT KO iPSC-CMs displayed no contractile force at all.

Amplitude and peak positions are present in both Ca²⁺ flux and AFM recordings and are directly comparable. Analysis was then extended to several key parameters of the cardiomyocyte contraction profile as previously analyzed in Ca²⁺ traces, contraction duration, amplitude, and decay time (tau). A direct correlation of the iPSC-CM beating force with increased pressure levels were observed.

Moreover, arrhythmic events in DCM iPSC-CMs versus WT controls were compared for AFM recordings with those detected in Ca2+ traces (FIGS. 6C-6K). Confirming comparability of this analysis method, levels arrhythmic events are comparable in Ca²⁺ and AFM measurements of DCM iPSC-CMs (FIGS. 6C-6K). In the WT controls, both AFM- and Ca²⁺ flux analysis did not detect a significant number of arrhythmic events.

AFM measurements for >1 h in DCM patient-specific and WT control iPSC-CMs were successfully recorded.

CONCLUSION: In this embodiment, complete curve fitting is possible for iPSC-CM Ca²⁺ traces and contractile force recordings. Moreover, automated calculations of signal on- and off-rates allow deeper analysis of disease-specific data sets such as from DCM iPSC-CMs, which experience higher noise variability

### Doctrine of Equivalents

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A method for processing signals from calcium transients comprising:
obtaining cardiomyocyte data comprising one dimensional trace data;
identifying a peak in the one dimensional trace data; and
fitting the peak to a shape function.

2. The method of claim 1, further comprising identifying peak features based on the shape functions.

3. The method of claim 1, wherein the cardiomyocyte data is obtained from at least one of the following: electrocardiography, fluorescence, and atomic force microscopy.

4. The method of claim 1, wherein the peaks are identified by a local maxima above a threshold.

5. The method of claim 4, wherein the threshold is set to 0.7*(Yₘₐₓ−Yₘᵢₙ)+Yₘᵢₙ.

6. The method of claim 1, wherein the fit shape function is a Voigt function.

7. The method of claim 6, wherein the shape function is fit using a Levenberg-Marquadt algorithm.

8. The method of claim 1, further comprising identifying a key peak feature from the shape function.

9. The method of claim 8, wherein the key peak feature is selected from the group consisting of peak start, peak 50%, 90% decay, peak 50% width, peak 90% width, maximum rising rate, maximum falling rate, pulse T₅₀, standard deviation of beat interval, standard deviation of beat rate, T₅₀, amplitude (ΔF/F₀), average maximum rising rate, and average maximum decaying rate.

10. A method to treat an individual based on cardiomyocyte function, comprising:
obtaining a cardiomyocyte from an individual;
treating the cardiomyocyte with a drug;
obtaining a measurement of cardiomyocyte function of the cardiomyocyte;
assessing treatment efficacy of the drug based on the measurement of cardiomyocyte function; and
treating the individual with the drug.

11. The method of claim 10, wherein the cardiomyocyte is an iPSC derived cardiomyocyte.

12. The method of claim 10, wherein the measurement of cardiomyocyte function is obtained from at least one of the following: electrocardiography, fluorescence, and atomic force microscopy.

13. The method of claim 10, wherein the assessing step is accomplished by:
identifying a peak in the one dimensional trace data; and
fitting the peak to a shape function.

14. The method of claim 13, wherein the peaks are identified by a local maxima above a threshold.

15. The method of claim 14, wherein the threshold is set to 0.7*(Yₘₐₓ−Yₘᵢₙ)+Yₘᵢₙ.

16. The method of claim 13, wherein the fit shape function is a Voigt function.

17. The method of claim 16, wherein the shape function is fit using a Levenberg-Marquadt algorithm.

18. The method of claim 13, wherein the assessing step further comprises identifying a key peak feature from the shape function.

19. The method of claim 18, wherein the key peak feature is selected from the group consisting of peak start, peak 50%, 90% decay, peak 50% width, peak 90% width, maximum rising rate, maximum falling rate, pulse T₅₀, standard deviation of beat interval, standard deviation of beat rate, T₅₀, amplitude (ΔF/F₀), average maximum rising rate, and average maximum decaying rate.

20. The method of claim 10, wherein the obtaining a measurement of cardiomyocyte function is a baseline measurement of cardiomyocyte function and further comprising obtaining a measurement of cardiomyocyte function of the treated cardiomyocytes.
